(19) United States
(12) Patent Application Publication
(10) Pub. No.: US 2008/0085244 A1
(43) Pub. Date: Apr. 10, 2008

(54) Methods for identifying and using amyloid-inhibitory compounds

(75) Inventors: Suzana Petanceska, Yonkers, NY (US); Sam Gandy, Philadelphia, PA (US); Donald E. Frail, Wildwood, MO (US)

Correspondence Address: Wyeth c/o Darby & Darby, P.C., P.O. BOX 770, Church Street Station, NEW YORK, NY, 10008-0770, US

(73) Assignees: Wyeth, Madison, NJ (US); Nathan S. Kline Research Institute, Orangeburg, NY (US)

(21) Appl. No.: 11/952,531

(22) Filed: Dec. 7, 2007

Related U.S. Application Data
(63) Continuation of application No. 09/695,446, filed on Oct. 24, 2000.
(60) Provisional application No. 60/163,819, filed on Nov. 5, 1999.

(51) Int. Cl. A61K 49/00; G01N 33/00

(52) U.S. Cl. 424/9.2; 435/7.92

(57) ABSTRACT

The present invention relates to identification of agents that pay a role in regulating brain amyloid-β (Aβ) levels in vivo. The invention provides compounds and methods of using such compounds to treat amyloidogenic conditions. It also provides a useful animal model for screening for and evaluating candidate amyloid inhibiting or therapeutic compounds. In particular, ovariectomy (ovx) and estrogen replacement were found to affect brain Aβ, levels in guinea pigs. Long-term ovx of guinea pigs resulted in increased levels of total brain Aβ, as compared to intact animals, and the Aβ42/Aβ40 ratio was also elevated. Treatment of ovx guinea pigs with β17-estradiol for ten days partially reversed the ovx-associated increase in brain Aβ levels.

### FIELD OF THE INVENTION

The present invention relates to identification of agents that play a role in regulating brain amyloid-P (Aβ) levels in vivo. The invention provides compounds and methods of using such compounds to treat amyloidogenic conditions. It also provides a useful animal model for screening for and evaluating candidate amyloid lowering or therapeutic compounds.

### BACKGROUND OF THE INVENTION

Alzheimer's Disease (AD) is a neurodegenerative disorder characterized by progressive deterioration of cognitive function and concomitant accumulation of parenchymal amyloid plaques, cerebrovascular amyloid deposits, intracellular neurofibrillary tangles, and loss of neurons and synapses (Tomlinson and Corsellis, Aging and the Dementias In: Greenfield's Neuropathology, Adams J H, Corsellis J A N, Duchen L W (eds); John Wiley & Sons, Inc., 1984, pp. 951-1025). In particular, there is dramatic degeneration of basal forebrain cholinergic neurons which project to the cerebral cortex and the hippocampus (Coyle, et al., Science, 1983, 219:1184-1190). The major component of these cerebral and cerebrovascular deposits is amyloid β (Aβ), a 40 or 42 amino acid, highly aggregable peptide, derived by proteolytic processing of the amyloid precursor protein (APP) (Selkoe, D. J., Trends Cell Biol., 1998, 8:447-53). Aβ42 is (thought to be) primarily responsible for the initial aggregation, in part due to a more hydrophobic character. Although the pathogenesis of AD is complex, a growing body of evidence indicates that the neuritic dystrophy, neurofibrillary tangle formation, gliosis, microglial reactivity, and other degenerative changes seen in AD brains are a result of altered metabolism of Aβ peptides (Selkoe, D. J., supra). Aβ peptides are generated by the action of β- (BACE; Vassar et al., Science, 1999, 286:735-741) and γ-secretase activities; in an alternative, non-amyloidogenic scenario, the generation of Aβ is precluded by the action of a third proteolytic activity, α-secretase. The secretase activities are under the control of numerous signal transduction pathways (Gandy, S., Trends Endocrinol. Metabol., 1999, 7:273-279).

The majority of AD (over 90%) is sporadic, and the identification of factors that influence the onset and/or progression of the disease would be an important step toward understanding its mechanism(s) and for developing successful, rational therapies. Along this line, compelling epidemiological evidence indicates that estrogen status may play an important role in the etiology of the disease: the prevalence of AD appears to be greater in women than in men (Mayeux and Gandy, Alzheimer's Disease, In: Women and Health Goldman M B and Hatch M C (eds), Academic Press, 1999), and postmenopausal women receiving estrogen replacement therapy (ERT) have a significantly delayed or reduced risk of developing AD (Tang et al., Lancet, 1996, 348:429-432; Kawas et al., Neurol., 1997, 48:1517-1521).

An avenue of recent research has been the investigation of the influence of estrogen on APP metabolism (Jaffe et al., J. Biol. Chem., 1994, 269:13065-13068; Kwan et al., Adv. Exp. Med. Biol., 1997, 429:261-271; Xu et al., Nat. Med., 1998, 4:447-451). Physiological concentrations of estrogen (17 β-estradiol, E2) decreased the levels of Aβ40 and Aβ42 peptides released from rodent or human primary neuronal (embryonic cerebral cortex) cultures (Xu et al., supra). In light of these findings, and since Aβ deposition appears to play a central role in initiating AD pathology, there is a need in the art to evaluate the ability of female gonadal hormone status to modulate brain Aβ levels in vivo. The in vitro results, while promising, are by no means predictive of in vivo effects.

In vivo, estrogen has been identified as having utility in treating adverse behavioral symptoms that accompany fluctuations in hormones associated with menopause in aging women, although the biochemical basis for these effects has never been determined. As such, the treatment of behavioral effects with estrogen in human subjects has been restricted to the treatment of menopause in women who demonstrate signs of deficiency in estrogen, and use in prevention of the sequelae of menopause, namely hot flashes and osteoporosis, which are typically corrected by replacement therapy of estrogen.

Although clinical studies by Sherwin (Psychoneuroendocrinology, 1988, 13:345-357), and Sherwin and Phillips (Annals of the New York Academy of Sciences, 1990, 592:474-5), have shown a general mood enhancing effect in oophorectomized women following intramuscular administration of estrogen at doses of 10 mg, the mechanism by which this effect occurred is unclear. In addition, these studies demonstrate that estrogen administered intramuscularly subsequently reaches the brain as inferred by the behavioral effects of the treatment and as predicted from the structure of the molecule.

Biochemical studies on the action of estrogen on cells of the CNS either in vivo or in vitro has resulted in conflicting reports. A number of studies have shown that estradiol has an effect on the plasticity of neurons. Morse et al. (Experimental Neurology, 1986, 94:649-658), reported that an estrogen derivative enhances sprouting of commissural-associational afferent fibers in the hippocampal dentate gyrus following entorhinal cortex lesions. Additionally, cyclic changes in synaptic density in the CA1 of the hippocampus were shown to be related to circulating E2 levels (Woolley et al., J. of Neurosci., 1992, 12:2549-2554) and these changes could be mimicked with exogenous E2 administration. Indeed, it has further been shown that ovariectomy reduces and E2 replacement normalizes high affinity choline uptake (HACU) in the frontal cortex of rats.

Additionally, Gibbs et al. (Soc. for Neurosci. Abstracts, 1993, 19:5) have reported upregulation of choline acetyltransferase (CHAT) levels following estradiol treatment in the medial septum after two days and two weeks of treatment, although no effect was observed after one week using in situ hybridization of ChAT mRNA. Luine et al. (Brain Res., 1980, 191:273-277), reported increased CHAT levels in the preoptic and hypothalamic regions of the rat brain in response to estradiol treatment.

U.S. Pat. No. 5,554,601 (Simpkins et al.) (the “'601 patent”) reports that estrogen compounds act on a fundamental process that impacts cell viability and cell response to adverse conditions that result in damage and death. An example of such conditions includes the regulation of glucose to cells. Administration of estrogen in a physiological dose results in the reversal of impairment of non-spatial learning in female rats that had been ovariectomized (ovx). These behavioral effects of short-term ovx and E2-replacement were correlated with biochemical changes in the hippocampus and the frontal cortex of the brain; in particular, a reduction and increase in high affinity choline uptake (HACU) in ovx and E2-controlled release pellet treated rats, respectively. Short-term E2-replacement also had a positive effect on choline acetyltransferase activity (ChAT) in the hippocampus, but not in the frontal cortex. Long-term E2 replacement appeared to prevent the time-dependent decline of ChAT in the frontal cortex and to attenuate CHAT activity decline in the hippocampus. Collectively, these data reportedly showed that estrogen has a cytoprotective effect on cells in the CNS and that the estrogen environment of adult female rats influences learning and the activity of basal forebrain cholinergic neurons. The data also demonstrated the importance of estrogens in the maintenance and proper function of basal forebrain cholinergic neurons in the female rat. The '601 patent lacks any indication that estrogens regulate APP processing and Aβ production.

This work establishes that estrogen has therapeutic effects on mood and on bone density in post-menopausal women, and appears to have protective effects on nervous system cells. However, there is no indication that estrogen can in any way affect amyloidosis, or that it regulates Aβ production in vivo. Thus, there is a need in the art to identify such compounds, and to develop animal models useful in screening for and testing of candidate compounds.

The present invention addresses these and other needs in the art.

### SUMMARY OF THE INVENTION

The present invention contemplates a method for reducing the level of amyloid-P (Aβ) peptides in vivo, where the method comprises administering an Aβ level reducing dose of an estrogen compound to an animal. In a further embodiment of the present invention, the Aβ peptides comprise Aβ42 and Aβ40, and the method further comprises reducing the ratio of Aβ42 to Aβ40.

In alternative embodiment of the invention, a method for evaluating the ability of a test compound to reduce the level of Aβ in vivo is contemplated. The method comprises comparing the level of Aβ of an orchidectomized non-human animal treated with the test compound to the level of Aβ in an orchidectomized non-human control animal, where a reduction of the level of Aβ in the animal treated with the test compound compared to the control animal indicates the ability of the test compound to reduce the level of Aβ in vivo. In a further embodiment of the inventions, the animal is an ovariectomized (ovx) animal. In a further embodiment, the test compound is an estrogen compound.

The present invention also contemplates a method for evaluating the ability of a test compound to reduce the level of Aβ in vivo. The method comprises comparing the level of Aβ of an ovx non-human animal selected from the group consisting of a guinea pig and a transgenic rodent that expresses human amyloid precursor protein treated with the test compound to the level of Aβ in an ovx non-human control animal, where a reduction of the level of Aβ in the animal treated with the test compound compared to the control animal indicates the ability of the test compound to reduce the level of Aβ in vivo.

The present invention further contemplates a method for evaluating the ability of a test compound to reduce the ratio of Aβ42 to Aβ40 in vivo. The method comprises comparing a ratio of Aβ42 to Aβ40 in an orchidectomized non-human animal treated with a test compound to the ratio of Aβ42 to Aβ40 in an orchidectomized non-human control animal, where a reduction of the ratio of Aβ42 to Aβ40 in the animal treated with the test compound compared to the control animal indicates the ability of the test compound to reduce the ratio of Aβ42 to Aβ40 in vivo. In a further embodiment, the animal is an ovariectomized (ovx) animal.

In another embodiment of the present invention, a method for reducing the level of Aβ in a subject to prevent the onset of or ameliorate a disease or disorder associated with amyloidosis is contemplated. The method comprises administering an Aβ level reducing dose of an estrogen compound to the subject. In a further embodiment, the estrogen compound is administered daily for at least ten days.

The present invention also contemplates a method for predicting the increased likelihood of amyloidosis in a subject. The method comprises observing a reduction in a level of an estrogen compound in the subject compared to a normal level or a level in the animal at an earlier time point. In a further embodiment, the estrogen compound is estrogen β17 or an aromatizable androgen. In an alternative embodiment, the amyloidosis comprises deposition of Aβ peptides. A further embodiment comprises predicting an increased likelihood of developing Alzheimer's disease.

### DESCRIPTION OF THE DRAWINGS

FIGS. 1A and 1B. Effect of ovariectomy and E2 treatment on serum estradiol levels (1A) and uterine weight (1B). Animal cells: i) intact guinea pigs (intact), ii) guinea pigs ovariectomized at 8 weeks of age and sacrificed 10 weeks later (ovx), and guinea pigs ovariectomized at 8 weeks of age and treated with iii) low-dose E2 (1 mg of E2/kg BW/day), or iv) high-dose E2 (5 mg of E2/kg BW/day) for 10 days. In each case, the E2 treatment began 8 weeks after ovariectomy. Horizontal lines indicate median values.

FIGS. 2A, 2B, and 2C. Effect of ovariectomy and E2 replacement on brain Aβ levels. Aβ40 and Aβ42 levels were determined by ELISA assays of DEA brain extracts. The total Aβ (A) and Aβ40 (B) values (an average of four readings) for each animal were normalized to brain tissue weight (g), and expressed as ng(Aβ)/g(wet weight). Horizontal lines indicate median values. (C) Aβ42 levels were calculated for each animal and a mean+/−SEM value was determined for each set of animals.

FIG. 3. Effect of ovariectomy and E2 treatment on sAPPα levels in brain. sAPPα levels were determined by quantitative Western blotting of DEA extracts using the 6E10 antibody standardized to corresponding flAPP values. For each group of animals, mean+/−SEM value was determined.

### DETAILED DESCRIPTION

The present invention advantageously establishes that treatment with female gonadal hormone agonists, and particularly with estradiol, affects Aβ levels in vivo, surprisingly without affecting soluble, APP levels. This invention is based, in part on the discovery of the effects of ovariectomy (ovx) and estrogen replacement on brain Aβ levels in guinea pigs. Long-term (10 weeks) ovx of guinea pigs resulted in increased levels of total brain Aβ (1.5-fold average increase, p<0.00001) as compared to intact animals. The Aβ42/Aβ40 ratio was also elevated (1.3-fold average increase, p<0.001). Treatment of ovx guinea pigs with E2 for ten days (beginning 8 weeks after ovx) partially reversed the ovx-associated increase in brain Aβ levels (20% average decrease; p<0.01). These data provide the first direct evidence that female gonadal hormone status plays a role in regulating brain Aβ levels in vivo.

In a preferred embodiment of the invention, female gonadal hormone status regulates Aβ42 levels more than Aβ40 levels. In this embodiment, a decrease in the level of estrogen increases the level of Aβ42 to greater extent then the level of Aβ40. Additionally, a decrease in the level of estrogen (ovx animals) increases the Aβ42/Aβ40 ratio compared to control animals. These data provide evidence that estrogen levels affect Aβ42 levels to a greater degree than Aβ40. The data also indicates that estrogen supplementation can at least partially offset this imbalance, leading to a decrease in the Aβ42/Aβ40 ratio.

A surprising discovery of the present invention is that the level of sAPPα does not change in response to administration of an estrogen compound. Thus, this marker of APP metabolism, which was monitored in in vitro assays of cultured primary and neuroblastoma cells for evidence of 17β-estradiol activity (see Xu et al, Nat. Med., 1998, 4:447), would not have yielded the discovery made herein: that estrogen compounds reduce Aβ levels in vivo. Indeed, the prior in vitro data supported a role of estrogen in increasing non-amyloidogenic processing by increasing the secretory metabolism of APP. The results disclosed here show that a change in sAPPα levels (up or down) is a poor guide to anti-amyloid drug development.

“Reducing a level of amyloid-P (Aβ) peptides” specifically refers to decreasing the amount of Aβ40 or, preferably, Aβ42, or more preferably, both, in vivo. Aβ can accumulate in blood, cerebrospinal fluid, or organs. The primary organ of interest for reducing the level of Aβ is brain, but Aβ levels may also be reduced in body fluids, tissues, and/or other organs by the practice of this invention.

As used herein, the term “about” or “approximately” means within 50% of a given value, preferably within 20%, more preferably within 10%, more preferably still within 5%, and most preferably within 1% of a given value. Alternatively, the term “about” or “approximately” means that a value can fall within a scientifically acceptable error range for that type of value, which will depend on how quantitative a measurement can be given the available tools.

### Estrogen Compounds

An “estrogen compound” is defined here and in the claims as any of the structures described in the 11th edition of “Steroids” from Steraloids Inc., Wilton N.H., here incorporated by reference. Included in this definition are non-steroidal estrogens described in the aforementioned reference. Other estrogen compounds included in this definition are estrogen derivatives, estrogen metabolites, estrogen precursors, selective estrogen receptor modulators (SERMs) and aromatizable androgens. The term also encompasses molecules that specifically trigger the estrogen effect described herein of decreasing the level of amyloid in vivo. Also included are mixtures of more than one estrogen or estrogen compound. Examples of such mixtures are provided in Table II of U.S. Pat. No. 5,554,601 (see column 6). Examples of estrogens having utility either alone or in combination with other agents are provided, e.g., in U.S. Pat. No. 5,554,601. In a specific embodiment, the estrogen compound is a composition of conjugated equine estrogens (PREMARIN™; Wyeth-Ayerst).

β-estrogen is the β-isomer of estrogen compounds. α-estrogen is the α-isomer of estrogen components. The term “estradiol” is either α- or β-estradiol unless specifically identified.

The term “E2” is synonymous with β-estradiol, 17β-estradiol, and β-E2. αE2 and α-estradiol is the α isomer of βE2 estradiol.

Preferably, a non-feminizing estrogen compound is used. Such a compound has the advantage of not causing uterine hypertrophy and other undesirable side effects, and thus, can be used at a higher effective dosage. Examples of non-feminizing estrogen include Raloxifene (Evista; Eli Lilly), Tamoxifen (Nolvadex; Astra Zeneca), and other selective estrogen receptor modulators.

Alternatively, a combination of an estrogen with a progestin, a combination of an estrogen with an anti-progestin, or a combination of estrogen with a non-feminizing estrogen may be used. Progestin compounds, for example, include progestins containing a 21-carbon skeleton and a 19-carbon (19-nortestosterone) skeleton.

In addition, certain compounds, such as the androgen testosterone, can be converted to estrogens in vivo by conversion with the aromatase enzyme. The aromatase enzyme is present in several regions including, but not limited to, the brain. Some androgens are substrates for aromatase and can be converted and some can not be a substrate. Those androgens that are substrates for aromatase are termed aromatizable androgens and those that are not substrates for aromatase are termed non-aromatizable androgens. Testosterone is, for example, an aromatizable androgen and dihydrotestosterone is, for example, a non-aromatizable androgen. Thus, the invention clearly extends to those compounds (and, as described infra, to using as test animals, animals in which the testes are removed or inactivated) that are converted from an androgen to an estrogen and that produces the effect described herein of decreasing the level of amyloid in vivo.

A “test compound” can be any molecule or combination of more than one molecule that affects amyloid production. The present invention contemplates screens for synthetic small molecule agents, chemical compounds, chemical combinations, and salts thereof as well as screens for natural products, such as plant extracts or materials obtained from fermentation broths. Other molecules that can be identified using the screens of the invention include proteins and peptide fragments, peptides, nucleic acids and oligonucleotides, carbohydrates, phospholipids and other lipid derivatives, steroids and steroid derivatives, prostaglandins and related arachadonic acid derivatives, etc. In a specific embodiment, the test compound can be an estrogen compound.

### Amyloid

The terms “amyloid,” “amyloid plaque,” and “amyloid fibril” refer generally to insoluble proteinaceous substances with particular physical characteristics independent of the composition of proteins or other molecules that are found in the substance. Amyloid can be identified by its amorphous structure, eosinophilic staining, changes in thioflavin fluorescence, and homogeneous appearance. Protein or peptide components of amyloid are termed herein “amyloid polypeptides,” and include, but are not limited to, β-amyloid peptide (Aβ), including synthetic βAPs corresponding to the first 28, 40, or 42 amino acids of Aβ, i.e., Aβ(1-28) or Aβ28, Aβ(1-40) or Aβ40, Aβ(1-42) or Aβ42, respectively, as well as a synthetic PAP corresponding to amino acids 25-35 of Aβ, i.e., Aβ₂₅₋₃₅. Other amyloid peptides include scrapie protein precursor or prion protein; immunoglobulin, including κ or λ light or heavy chains, or fragments thereof, produced by myelomas; serum amyloid A; β₂-microglobulin; apoA1; gelsolin; cystatin C; (pro)calcitonin; atrial natururetic factor; islet amyloid polypeptide, also known as amylin (see, Westermark et al., Proc. Natl. Acad. Sci. USA 84:3881-85, 1987; Westermark et al., Am. J. Physiol. 127:414-417, 1987; Cooper et al., Proc. Natl. Acad. Sci. USA 84:8628-32, 1987; Cooper et al., Proc. Natl. Acad. Sci. USA 85:7763-66, 1988; Amiel, Lancet 341:1249-50, 1993); and the like. In a specific aspect, the term “amyloid” is used herein to refer to substances that contain Aβ. “Amyloidosis” refers to the in vivo deposition or aggregation of proteins to form amyloid plaques or fibrils.

The 42 amino acid (4.2 kDa) beta-Amyloid Peptide (βAP) derives from a family of larger Amyloid Peptide Precursor (APP) proteins (Glenner and Wong, 1984, Biochem. Biophys. Res. Commun. 120:885-890; Glenner and Wong, 1984, Biochem. Biophys. Res. Commun. 122:1131-35; Goldgaber et al., 1987, Science 235:8778-8780; Kang et al., 1987, Nature 325:733-736; Robakis et al., 1987, Proc. Natl. Acad. Sci. USA 84:4190-4194; Tanzi et al., 1987, Science 235:880-884). APP is a transmembrane protein found in a number of isoforms, which in general are referred to herein as full length APP (flAPP). In addition, there is a soluble form of APP (sAPPα), formed by the action of a-secretase (discussed supra).

The “level of Aβ” in a biological sample can be detected by any method known in the art, including by not limited to immunoassay (as exemplified infra), biochemical analysis (e.g., purification, gel electrophoresis, quantitative amino acid sequence analysis or composition analysis, Congo red or Thioflavin-T staining, and the like), or other methods known to detect Aβ. In particular, fluorescence methods using Thioflavin T are used to detect aggregated peptide. A “biological sample” includes, but is not limited to body fluids (blood, blood cells, plasma, serum, cerebrospinal fluid, urine), tissues (e.g., spinal chord, nerves, etc.), or organs (preferably brain, but also including liver, kidney, pancreas, etc.).

A disease or disorder is associated with amyloidosis when amyloid deposits or amyloid plaques are found in or in proximity to tissues affected by the disease, or when the disease is characterized by overproduction of a protein, particularly an amyloid protein, that is or can become insoluble. The amyloid plaques may provoke pathological effects directly or indirectly by known or unknown mechanisms. Examples of amyloid diseases include, but are not limited to, systemic diseases, such as chronic inflammatory illnesses, multiple myeloma, macroglobulinemia, familial amyloid polyneuropathy (Portuguese) and cardiomyopathy (Danish), systemic senile amyloidosis, familial amyloid polynephropathy (Iowa), familial amyloidosis (Finnish), Gerstmann-Straussler-Scheinker syndrome, familial amyloid nephropathy with urticaria and deafness (Muckle-Wells syndrome), medullary carcinoma of thyroid, isolated atrial amyloid, and hemodialysis-associated amyloidosis (HAA); and amyloid associated neurodegenerative diseases.

As noted above, in addition to systemic amyloidosis, the present invention relates particularly to neurodegenerative diseases involving amyloidosis. The term “neurodegenerative disease” refers to a disease or disorder of the nervous system, particularly involving the brain, that manifests with symptoms characteristic of brain or nerve dysfunction, e.g., short-term or long-term memory lapse or defects, dementia, cognition defects, balance and coordination problems, and emotional and behavioral deficiencies. Such diseases are “associated with amyloidosis” when histopathological (biopsy) samples of brain tissue from subjects who demonstrate such symptoms would reveal amyloid plaque formation. As biopsy samples from brain, especially human brain, are obtained with great difficulty from living subjects or might not be available at all, often the association of a symptom or symptoms of neurodegenerative disease with amyloidosis is based on criteria other than the presence of amyloid deposits, such as plaques or fibrils, in a biopsy sample. Thus, particularly with respect to AD, traditional diagnosis depends on symptomology and, if relevant, family history. In clinical practice a physician will diagnose Alzheimer's Disease on the basis of symptoms of senile dementia, including cognitive dysfunction, retrograde amnesia (loss of memory for recent events), progressive impairment of remote memory, and possibly depression or other neurotic syndromes. The individual presents with slow disintegration of personality and intellect. Imaging may reveal large cell loss from the cerebral cortex and other brain areas. AD differs from senile dementia, however, by age of onset: AD is likely to occur in the fifth or sixth decade, whereas senile dementia occurs in the eighth decade or later.

In a specific embodiment, according to the present invention the neurodegenerative disease associated with amyloidosis is Alzheimer's disease (AD), a condition that includes, sporadic AD, ApoE4-related AD, other mutant APP forms of AD (e.g., mutations at APP717, which are the most common APP mutations), mutant PS1 forms of familial AD (FAD) (see, WO 96/34099), mutant PS2 forms of FAD (see, WO 97/27296), and alpha-2-macroglobulin-polymorphism-related AD. In other embodiments, the disease may be the rare Swedish disease characterized by a double KM to NL mutation in amyloid precursor protein (APP) near the amino-terminus of the PAP portion of APP (Levy et al., 1990, Science 248:1124-26). Another such disease is hereditary cerebral hemorrhage with amyloidosis (HCHA or HCHWA)-Dutch type (Rozemuller et al., 1993, Am. J. Pathol. 142:1449-57; Roos et al., 1991, n Ann. N.Y. Acad. Sci. 640:155-60; Timmers et al., 1990, Neurosci. Lett. 118:223-6; Haan et al., 1990, Arch. Neurol. 47:965-7). Other such diseases known in the art and within the scope of the present invention include, but are not limited to, sporadic cerebral amyloid angiopathy, hereditary cerebral amyloid angiopathy, Down's syndrome, Parkinson-dementia of Guam, and age-related asymptomatic amyloid angiopathy (see, e.g., Haan and Roos, 1990, Clin. Neurol. Neurosurg. 92:305-310; Glenner and Murphy, 1989, N. Neurol. Sci. 94:1-28; Frangione, 1989, Ann. Med. 21:69-72; Haan et al., 1992, Clin. Neuro. Neurosurg. 94:317-8; Fraser et al., 1992, Biochem. 31:10716-23; Coria et al., 1988, Lab. Invest. 58:454-8). The actual amino acid composition and size of the PAP involved in each of these diseases may vary, as is known in the art (see above, and Wisniewski et al., 1991, Biochem. Biophys. Res. Commun. 179:1247-54 and 1991, Biochem. Biophys. Res. Commun. 180:1528 [published erratum]; Prelli et al., 1990, Biochem. Biophys. Res. Commun. 170:301-307; Levy et al., 1990, Science 248:1124-26).

The instant invention contemplates evaluating amyloidogenic peptide from any animal, and more preferably, mammal, including humans, as well as mammals such as monkeys, dogs, cats, horses, cows, pigs, sheep, goats, rabbit, guinea pigs, hamsters, mice and rats.

### Animal Models

A “non-human animal” can be any animal, including without limitation a rodent (mouse, rat, guinea pig, hamster), rabbit, cat, dog, pig, goat, sheep, monkey (or other primate), horse, cow, etc. Typically, for ease of use in the laboratory, the non-human animal will be a small mammal, such as a rat, mouse, hamster, guinea pig, etc. The non-human animal may be transgenic. Preferably, such a transgenic non-human animal expresses a human APP or a human APP variant. In a preferred embodiment, the transgenic animal is a mouse or rat that is double transgenic and expresses human APP and a human presenilin protein or presenilin variant, e.g., PS-1 or PS-2, preferably PS-1. In a preferred embodiment, exemplified infra, the animal is an ovariectomized female guinea pig.

A “control animal” is an animal that is not treated with a test compound, or that is treated with a placebo compound that lacks amyloid-inhibitory activity.

The term “orchidectomized” refers to an animal that has had its gonads removed or ablated. Removal generally refers to surgical resection. Ablation refers to chemical treatment to destroy gonad function, radiation treatment, or some other method that results in destruction or dysfunction of the gonad. An “intact” animal has not been orchidectomized; preferably the gonads function normally in an intact animal. “Gonads” are the ovaries in females and testicles in males. In a preferred aspect of the invention the animal is “ovariectomized”, i.e., its ovaries are removed or ablated (such an animal must, of course, be a female).

### Transgenic Animals

As noted above, transgenic animals (Guenette and Tanzi, Neurobiol. Aging, 1999, 20:201-11), particularly orchidectomized transgenic animals, can be used in the practice of the invention. Games et al. (Nature, 1995, 373:523-7) described a transgenic mouse that expressed a human APP variant (APP with a phenylalanine for valine substitution at position 717) that progressively developed the hallmarks of AD. Other transgenic mice have also been described (Shen and Li, Brain Res Bull, 1998, 46:233-6 [expressing mRNAs for presenilin-1 and amyloid precursor protein (APP-695) from same neuronal populations in rat hippocampus]; Holcomb et al., Nat Med, 1998, 4:97-100 [accelerated Alzheimer-type phenotype in transgenic mice carrying both mutant amyloid precursor protein and presenilin 1 transgenes]; Borchelt et al., Neuron 1996 November; 17:1005-13 [familial Alzheimer's disease-linked presenilin 1 variants elevate Aβ1-42/1-40 ratio in vitro and in vivo]). In addition to APP and PS transgenic animals, ApoE transgenic animals are also of interest, particularly mice with the ApoE4 variant, which is associated with increased likelihood of developing AD.

Presenilins, and particularly mutant presenilins associated with familial Alzheimer's disease and thus desirable to transfer into transgenic animals, are described in International Patent Publication Nos. WO-96/34099, WO 97/27298, and WO 98/01549; see Annu Rev Neurosci, 1998, 21:479-505 (PS1, PS2, ApoE4, and other mutant proteins associated with AD, and their use in transgenic animals, are discussed).

### Prognosis and Diagnosis of Amyloidosis

A reduction in the levels of an estrogen compound in vivo results in increased amyloid production. This observation establishes the ability to predict whether a given subject will have an increased likelihood of developing amyloid deposits, and thus an increased likelihood of developing a disease or disorder associated with amyloidosis, e.g., Alzheimer's Disease. These predictions are based on observing a decrease in the level of the estrogen compound in the subject.

The term “increased likelihood” means that there is a greater probability of the specified outcome, e.g., amyloidosis, in a given individual. Since the actual development of the outcome depends on a number of factors, the actual course an individual will follow is unknowable. Thus, the present invention directs itself to probabilities and changes in probabilities.

A “decrease in the level” of an estrogen compound means that the amount or concentration of the compound in blood is lower than a normal level for that species or than in the subject at an earlier time. A “normal level” is a mean, median, or mode found in a population selected at random for testing.

The term “estrogen compound” has been defined above. Thus, the invention contemplates measuring levels of endogenous estrogen compounds (such as, but by no means limited to, E2, aromatizable androgens, or therapeutic estrogen compounds).

Testing for the level of the estrogen compound in a biological sample from a subject can be made using standard techniques. A “biological sample” is any body tissue or fluid likely to contain the estrogen compound. Such samples preferably include blood or a blood component (serum, plasma). The standard testing methods include immunoassay, biochemical assay, analytic testing (such as gas chromatography or mass spectrometry), and the like.

### Pharmaceutical Compostions and Administration

The estrogen compounds of the invention can be formulated in a pharmaceutical composition with a pharmaceutically acceptable carrier. The concentration or amount of the estrogen, progestin, anti-progestin, non-feminizing estrogen, or aromatizable androgen compound will depend on the desired dosage and administration regimen, as discussed below. The pharmaceutical compositions may also include other biologically active compounds, including but by no means limited to, androgens, anabolic hormones, non-steroidal anti-inflammatory drugs, immunomodulatory drugs, etc. In a specific embodiment, the compositions do not include androgens or anabolic hormones (and, indeed, in a related specific embodiment, such compounds are not administered with the estrogen compounds).

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

A composition comprising “A” (where “A” is a single protein, DNA molecule, vector, recombinant host cell, etc.) is substantially free of “B” (where “B” comprises one or more contaminating proteins, DNA molecules, vectors, etc.) when at least about 75% by weight of the proteins, DNA, vectors (depending on the category of species to which A and B belong) in the composition is “A”. Preferably, “A” comprises at least about 90% by weight of the A+B species in the composition, most preferably at least about 99% by weight. It is also preferred that a composition, which is substantially free of contamination, contain only a single molecular weight species having the activity or characteristic of the species of interest.

According to the invention, the estrogen compound formulated in a pharmaceutical composition of the invention can be introduced parenterally, transmucosally, e.g., orally (per os), nasally, or rectally, or transdermally. Parental routes include intravenous, intra-arteriole, intramuscular, intradermal, subcutaneous, intraperitoneal, intraventricular, and intracranial administration. Preferably, administration is oral.

In another embodiment, the therapeutic compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.). To reduce its systemic side effects, this may be a preferred method for introducing the compound.

In yet another embodiment, the therapeutic compound can be delivered in a controlled release system. For example, a polypeptide may be administered using intravenous infusion with a continuous pump, in a polymer matrix such as poly-lactic/glutamic acid (PLGA), a pellet containing a mixture of cholesterol and the estrogen compound (SilasticR™; Dow Corning, Midland, Mich.; see U.S. Pat. No. 5,554,601) implanted subcutaneously, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Ê Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Preferably, a controlled release device is introduced into a subject in proximity of the site of amyloidosis. Other controlled release systems are discussed in the review by Langer (Science 249:1527-1533 (1990)).

### Dosage and Regimen

A constant supply of the estrogen compound can be ensured by providing a therapeutically effective dose (i.e., a dose effective to induce metabolic changes in a subject) at the necessary intervals, e.g., daily, every 12 hours, etc. These parameters will depend on the severity of the disease condition being treated, other actions, such as diet modification, that are implemented, the weight, age, and sex of the subject, and other criteria, which can be readily determined according to standard good medical practice by those of skill in the art. Preferably, the estrogen compound is administered for at least ten days, more preferably at least 100 days, and more preferably still, for the life of the recipient.

The term “prevent the onset of” means to prophylactically interfere with a pathological mechanism that results in the disease or disorder. In the context of the present invention, such a pathological mechanism can be an increase in processing of the amyloidogenic form of APP; dysregulation of Aβ clearance; or some combination of the two. The term “ameliorate” means to cause an improvement in a condition associated with the disease or disorder. In the context of the present invention, amelioration includes a reduction in the level of Aβ, regulation of the formation of Aβ, decrease in aggregation of Aβ or the formation of amyloid plaques, or improvement of a cognitive defect in a subject suffering from a disease or disorder associated with amyloidosis, e.g., Alzheimer's disease or an animal model of Alzheimer's disease. The phrase “therapeutically effective amount” or “dose” is used herein to mean an amount or dose sufficient to reduce the level of amyloid peptide, e.g., by about 10 percent, preferably by about 50 percent, and more preferably by about 90 percent. Preferably, a therapeutically effective amount can ameliorate or prevent a clinically significant deficit in the activity, function, and response of the host. Alternatively, a therapeutically effective amount is sufficient to cause an improvement in a clinically significant condition in the host.

A subject who “has an increased risk of developing” a disease or disorder associated with amyloidosis may have a genetic predisposition to developing an amyloidosis, such as a person from a family that has members with familial Alzheimer's Disease (FAD). Alternatively, someone in his or her seventh or eighth decade is at greater risk for age-related AD.

A subject who “shows a symptom of” a disease or disorder associated with amyloidosis presents with a symptom or complaint found in subjects who have or have had such a disease or disorder. For example, in Alzheimer's Disease, these symptoms can include development of dementia, memory defects, and the like in the fifth and sixth decade, as discussed above.

An “Aβ level reducing dose” is an amount of estrogen compound that causes a decrease in the level of Aβ, e.g. as set forth above for a test animal. Depending on whether the recipient is a human, an animal in need of treatment, or an experimental animal, dosages can range from about 0.5 μg estrogen per kg body weight to (μg/kg) to about 50 mg/kg, per day; preferably from about 5 μg/kg to about 10 mg/kg, per day. The amount of estrogen compound used to decrease the level of Aβ can be an amount corresponding to the level of estrogen in a fertile female animal of the same species as the animal receiving the estrogen compound. Physiological activity of estrogen is well known and can be determined. A “fertile animal” or “intact animal” is an animal that has not been orchidectomized, and more specifically that has not been ovariectomized.

An “amount corresponding to the level” means that the concentration of the estrogen compound has the same activity as a pharmacological concentration of estrogen.

Various specific dosages are contemplated. While the 1 mg/kg and 5 mg/kg doses administered to guinea pigs in the Examples, infra, are very high, as noted above such dosages may be acceptable in animal models. Generally, as noted above, the minimum dosage is one that is effective to induce a reduction in the level of amyloid peptide. The maximum dosage is one that is tolerated by the recipient without experiencing undue side effects.

In a specific embodiment, when the estrogen compound is a composition of conjugated equine estrogens, such as PREMARIN™, the dosage can range from about 0.300 mg/kg/day to about 2.5 mg/kg/day in human patients. Typical dosages are 0.3 mg, 0.625 mg, 1.25 mg, and 2.5 mg. As discussed above, an equally effective amount of a different estrogen compound can be used.

In another specific embodiment, the estrogen compound is a non-feminizing estrogen, which can be administered at much higher dosages because it does not cause undesirable side effects. In this embodiment, the dosage can range from about 0.500 mg/kg to about 100 mg/kg, preferably up to about 50 mg/kg, and more preferably from about 10 mg/kg to 40 mg/kg. In a specific embodiment, the non-feminizing estrogen compound is Raloxifene. In another specific embodiment, combinations of an estrogen with a progestin, an estrogen with an F anti-progestin, and an estrogen with a non-feminizing estrogen also may be used.

A subject in whom administration of the estrogen compound is an effective therapeutic regiment for a disease or disorder associated with amyloidosis is preferably a human, but can be any animal, including a laboratory animal in the context of a clinical trial or screening or activity experiment. Thus, as can be readily appreciated by one of ordinary skill in the art, the methods and compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

### EXAMPLES

The present invention will be better understood by reference to the following examples, which are provided as illustrative of the invention and not by way of limitation.

### Example 1

### Ovariectomy and 17β-estradiol Modulates the Levels of Amyloid β Peptides in Brain

This Example shows that estrogen positively impacts amyloid-P levels, and provides an ovariectomized guinea pig model that provides for evaluation of drugs for treating Aβ formation.

### Materials and Methods

Maintenance of animals and treatment. Ovariectomized (ovx) and intact female guinea pigs were purchased from Hilltop Laboratories (Scottsdale, Pa.); ovx animals were 8 weeks old at the time of surgery. 17β-estradiol (E2) was purchased from Sigma (St. Louis, Mo.). Throughout the study, the animals were fed ad libitum in a controlled lighting environment (12 h/12 h light/dark cycles). After the surgery, the ovx guinea pigs were put on a casein-based, soy-free diet (Purina, Richmond, Ind.) to exclude the presence of phytosteroids in the diet. The intact animals also began receiving soy-free food at approximately 8 weeks of age. After 8 weeks on soy-free diet, the animals were divided into four groups: i) intact (n=8), ii) ovx (n=9), iii) ovx+low-dose E2 treatment (1 mg E2/kgBW) (n=9), and iv) ovx+high-dose E2 treatment (5 mg E2/kgBW) (n=8) (kgBW is kilograms of body weight). E2 was administered per os by powdering the hormone into the soy-free chow. Prior to the beginning of the treatment, all animals were weighed. The average daily food intake for each animal using this particular diet was determined in a preliminary experiment. The animals received soy-free food (intact and ovx groups) or soy-free food supplemented with E2 (ovx+low-dose E2 treatment, and ovx+high-dose E2-treatment) for 10 days.

Tissue collection. At the end of the treatment, all animals were sacrificed by decapitation. Trunk blood was collected for determination of E2 levels in the serum by radioimmunoassay (Diagnostic Products Laboratory). Uteri were removed and weighed to establish E2-induced hypertrophy. The brains were immediately removed, and the cerebellum was dissected away from each brain. The rest of the brain was divided into hemispheres which were snap-frozen and stored at −80° C.

Preparation of Brain Extracts. Soluble Proteins from the Brains were Recovered using a modification of an established protocol (Savage et al., J. Neurosci., 1998, 18:1743-52). Briefly, the hemispheres were homogenized in 0.2% diethylamine (DEA)/50 mM NaCl at 1:10 w/v ratio, with 5-6 strokes of a Dounce homogenizer. The DEA homogenate was centrifuged for 90 min at 100,000 g. The DEA supernatants were neutralized to pH about 8.0 by addition of 1/10th vol. of 0.5M Tris-Cl pH 6.8, then aliquoted and snap-frozen. The pellets of the DEA extracts were solubilized in 2% SDS/PBS containing a cocktail of protease inhibitors (“Complete”, Boehringer Mannheim, Germany), sonicated and boiled. The protein concentrations of the DEA and SDS supernatants were determined using the BCA reagent assay kit (Pierce, Rockford, Ill.).

Detection of sAPPα, flAPP, Aβ40 and Aβ42. The amino acid sequence of APP from guinea pigs is 97% identical to the human APP homologue and the Aβ region is 100% identical to human Aβ (Beck et al., Biochim. Biophys. Acta, 1997, 1351:17-21), thus enabling use of well characterized Aβ antibodies to study the effects of estrogen on APP metabolism.

Soluble APPα (sAPPα) was detected by Western blotting of proteins from the DEA extracts using the monoclonal antibody 6E10 (Senetek, St. Louis, Mo.), which recognizes residues 5-10 from the Aβ region. The DEA extraction recovers soluble and not membrane embedded proteins, precluding the interference of flAPP with the detection of sAPPα (Savage et al., supra).

For detection of the effect of E2 on the levels of sAPPα, Western blotting using 6E10 to detect this species was performed on triplicate samples from DEA extracts of each brain (50 μg/lane). Visualization was performed using enhanced chemiluminescence. For quantitation, multiple exposures of the immunoblots were scanned using the ScanAnalysis software. The average values (in densitometric units) for each sample were then standardized to the values obtained for flAPP. Full-length APP levels were determined by immunoblotting of SDS extracts (50 μg/lane) using antibody 369 (which recognizes epitopes in the cytoplasmic tail of APP, residues 645-695; Buxbaum et al., Proc. Natl. Acad. Sci. USA, 1990, 87:6003-6006). Again, the samples were analyzed in triplicate, and densitometric analysis of multiple exposures of the immunoblots was performed.

The levels of Aβ40 and Aβ42 were determined by Aβ40- and Aβ42-specific ELISA assays (Mehta et al, Neurosci. Lett., 1998, 241:13-16). For each animal, the levels of Aβ40, Aβ42, and total Aβ were standardized to brain tissue weight and expressed as ng (Aβ)/g (brain tissue, wet weight). In all experiments, animals were coded prior to tissue collection, and the treatment status of each animal was unknown to the investigators at the time of the assays.

Statistical analysis. For each analyzed parameter, the values obtained for the intact group or either of the ovx+E2 groups were compared to the values obtained for the ovx animals using a one tailed Student's t-test. The differences in total Aβ levels: i) between the intact group and the ovx group, ii) between the ovx group and the low-dose E2 group, and iii) between the ovx group and the high-dose E2 group, were also assessed using the Mann-Whitney nonparametric test.

### Results

Initially, the effect of orally administered 17β-estradiol (E2) on brain Aβ levels in ovariectomized (ovx) guinea pigs was evaluated. Seven ovx guinea pigs (8 weeks old at the time of ovx) were used. After ovx, the animals were fed soy-free, casein-based diet to avoid the consumption of estrogenic phytosteroids. Eight weeks following ovx, the animals were divided into two experimental sets: ovx group (n=3), and ovx+E2 group (1 mg E2/1 kg body weight (BW)/day; n=4). E2 was administered per os by powdering the hormone into the soy-free chow. The ovx+E2 animals were treated for 10 days. After the treatment, all animals were sacrificed, and blood, uteri, and brains were collected for analysis. Uterine weights and serum E2 levels were determined to document the hormonal status. The levels of Aβ40, Aβ42, and sAPPα in brain tissue were determined using Aβ40- and Aβ42-specific ELISA assays and quantitative immunoblotting, respectively, as described in Methods.

As expected, the 10-day oral administration of E2 led to uterine hypertrophy and a dramatic increase in serum E2 levels in the ovx+E2 group as compared to the ovx group (greater than 3-fold increase in uterine weight, p=0.0003, and greater than 5-fold increase in serum E2 levels, p<0.00001). In addition, the E2 treatment appeared to correlate with decreased levels of brain Aβ (20% average decrease in total Aβ levels), approaching statistical significance (p=0.09). The levels of sAPPα were indistinguishable between the ovx group and ovx+E2 group (p=0.5).

A second experiment was conducted, aimed at investigating the effect of long-term (10 weeks) ovx on brain Aβ levels as well as the effect of short-term (10 days) E2 replacement on Aβ in the brains of ovx animals using low or high doses of E2. This extended study employed 4 groups of animals: intact group (n=8), ovx group (n=9), ovx+low E2 group (1 mg/kgBW/day) (n=9), and ovx+high E2 group (5 mg/kgBW/day) (n=8). The treatments (ovx+E2) were performed as in the first experiment: 8 weeks after ovx, the chow of the ovx+low E2 and the ovx+high E2 animals was supplemented with E2 for 10 days. At the end of the E2 treatment, all animals were sacrificed by decapitation, and blood, uteri, and brains were isolated and subjected to analysis.

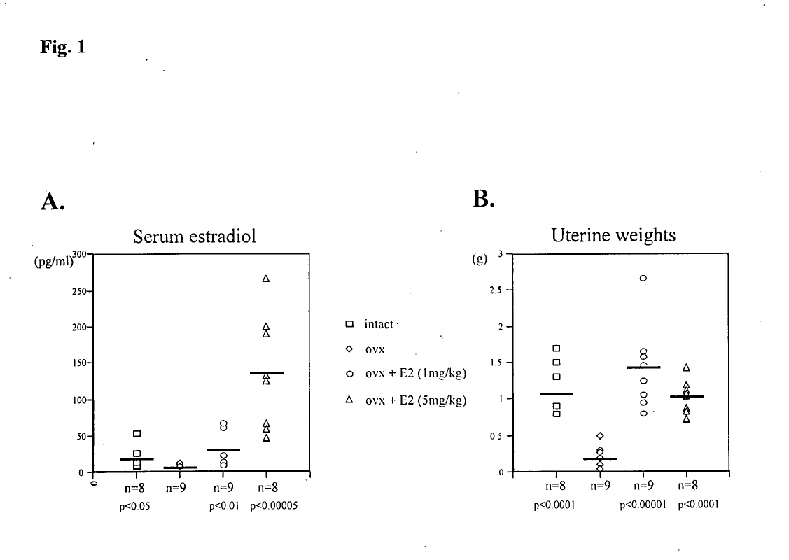

Long-term ovx was associated with a decrease in serum E2 levels as compared to age-matched, intact animals (FIG. 1A; Table). Ten days of replacement with low dose E2 (1 mg E2/kg BW/day) or high dose E2 (5 mg E2/kg BW/day) led to dose-dependent increases in serum E2 levels when compared to either the ovx group or to the intact group (FIG. 1A; Table). The values for serum E2 levels of the intact animals varied: some were comparable to the serum E2 levels of ovx animals, while others were comparable to the serum E2 levels of ovx+low-dose E2 animals. This variation is typical of the normal asynchronous cycling of intact animals (Shi et al., Biol. Reprod., 1999, 60:78-84). The high-dose E2 treatment resulted in supraphysiological levels of serum E2 (FIG. 1A; Table).

TABLE
Median and mean +/− SEM values for plasma E2 levels, uterine weights, total Aβ levels
and Aβ42/Aβ40 ratios of the intact, ovx, ovx + low-dose E2 groups.

| Table | intact | ovx | ovx + low-dose E2 | ovx + high-dose E2 |
|---|---|---|---|---|
| number of animals | 8 | 9 | 9 | 8 |
| Serum E2 (pg/ml) median | <7.6 | 9.2 | 21.8 | 128.8 |
| mean +/− SEM | <7.6 | 17 +/− 5.7 | 25.7 +/− 7.3 | 135.9 +/− 27.7 |
| p (one tailed Student's test) | p < 0.05 | | p < 0.01 | p < 0.0005 |
| Uterine weight (g) median | 0.9 | 0.2 | 1.37 | 1.055 |
| mean +/− SEM | 1.1 +/− 0.12 | 0.227 +/− 0.04 | 1.4 +/− 0.18 | 1.03 +/− 0.08 |
| p (one tailed Student's test) | p < 0.0001 | | p < 0.00001 | p < 0.0001 |
| Total Aβ (ngAβ/g brain tissue) | 1.568 | 2.391 | 2.063 | 2.094 |
| (median) | | | | |
| mean +/− SEM | 1.608 +/− 0.48 | 2.456 +/− 0.04 | 2.023 +/− 0.134 | 1.998 +/− 0.175 |
| p (one tailed Student's test) | p < 0.0001 | | p < 0.01 | p = 0.014 |
| p (Mann Whitney test) | p < 0.00001 | | 0.025 < p < 0.01 | p < 0.025 |
| Aβ42/Aβ40 ratio median (range) | 0.120 | 0.154 | 0.146 | 0.140 |
| mean +/− SEM | 0.119 +/− 0.005 | 0.150 +/− 0.003 | 0.141 +/− 0.01 | 0.141 +/− 0.013 |
| p (one tailed Student's test) | p < 0.001 | | p = 0.21 | p = 0.25 |

The uteri of the ovx animals were hypotrophic when compared to the uteri of the intact group of animals: on average, uteri from ovx animals weighed less than one third that of uteri from intact animals (FIG. 1B; Table). The uteri of the ovx animals that had received low-dose E2 for 10 days were hypertrophied and had weights comparable to, or higher than, those of the intact group (FIG. 1B). High-dose E2 treatment was also associated with uterine hypertrophy, though the uterine weights did not exceed those of the ovx+low-dose E2 group (FIG. 1B; Table).

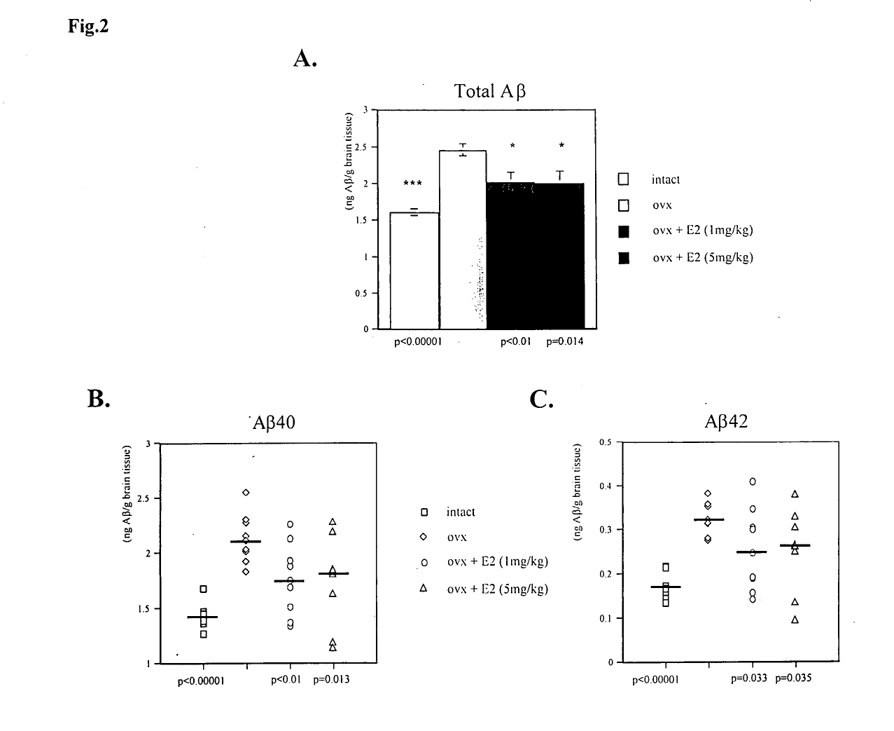

The 10-week ovx was associated with increased levels of brain Aβ as compared to intact animals (1.5-fold average increase in total Aβ; p<0.0001) (FIG. 2A; Table). It is of note that the levels of Aβ42 increased to a greater extent than the levels of Aβ40 (1.8-fold average increase for Aβ42; p<0.0001, and 1.5-fold average increase for Aβ40; p<0.00001) (FIGS. 2B, 2C). This resulted in an increased Aβ42/Aβ40 ratio in the ovx group as compared to the intact group (1.3-fold average increase; p<0.001) (Table).

Treatment of ovx guinea pigs with the low-dose E2 for 10 days, beginning 8 weeks after ovx, was associated with partial reversal of the ovx-induced elevation of total brain Aβ levels (18% average decrease; p<0.01) (FIG. 2A and Table). Aβ40 and Aβ42 levels decreased to a similar extent (18% average decrease for Aβ40, p<0.01; 21% average decrease for Aβ42, p=0.033) (FIGS. 2B, 2C). The high-dose E2 treatment (5 mg/kg BW/day) had a similar effect, and did not cause any additional decrease in either Aβ species (FIG. 2; Table). Interestingly, in few individual animals receiving E2 in either E2-treatment group, the levels of brain Aβ were similar to, or lower than, those observed in animals from the intact group (FIGS. 2B, 2C). The 10-day E2 treatment (both low and high-dose) did not alter the Aβ42/Aβ40 ratio on average (Table). However it is of note that the Aβ42/Aβ40 for few individual animals from the E2 treatment groups was comparable to the ratio observed in animals from the intact group.

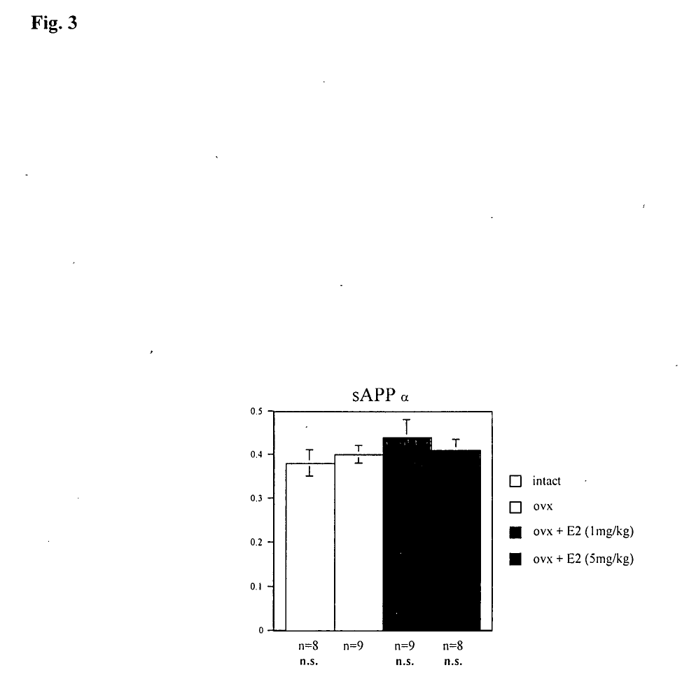

The levels of sAPPα were unaffected by ovx or E2 replacement (FIG. 3). This effect on sAPPα is in contrast with data from cell culture studies where the estrogen-induced decrease in Aβ peptides was accompanied by an increase in sAPPα levels in the cell culture media (Xu et al., Nat. Med., 1998, 4:447-51). Similar to our findings, and also in contrast to cell culture studies, the sAPPα levels remained unchanged in response to treatment with phorbol ester in vivo (Savage et al., J. Neurosci., 1998, 18:1743-52). This suggests that in brain in vivo, the reciprocal relationship between Aβ peptide and sAPPα release that has been observed in cultured cells may be less evident or absent.

### Discussion

These are the first data indicating that the levels of Aβ in brain are under the control of gonadal hormones. More specifically, we present evidence that prolonged ovariectomy is associated with increased brain Aβ40 and Aβ42 levels in vivo, and that this increase can be at least partially reversed by E2 replacement for 10 days. These data further indicate that the ratio of Aβ42 to Aβ40 differs between ovx guinea pigs and control animals and that the levels of Aβ42 increased to a greater extent than the levels of Aβ40, resulting in an increase in the Aβ42/Aβ40 ratio. This suggests that Aβ42 formation is regulated by a estrogen to a greater extent than the formation of Aβ40. Moreover, E2 replacement may offset this imbalance (reducing the mean Aβ42/Aβ40 ratio from 0.15 to 0.141), although the statistical difference of these data was p=0.25. Therefore, ovx guinea pigs represents a useful animal model for evaluating the impact of estrogen and “designer” estrogen-like compounds on brain Aβ metabolism in vivo.

Since our studies involved assays of steady state levels of APP metabolites in response to ovariectomy and E2 replacement, we were unable to distinguish whether the changes in Aβ levels reflected altered Aβ generation or altered Aβ clearance. Also, it remains to be determined whether the observed effects on Aβ metabolism occur in response to activation of brain estrogen receptors or whether they are mediated by estrogen receptor-independent mechanisms.

Cessation of ovarian estrogen production in postmenopausal women might facilitate Aβ deposition by increasing the local concentrations of Aβ40 and Aβ42. The results of a related study on plaque-forming transgenic mice, showing that prolonged ovx accelerates the elevation of brain Aβ levels, support this hypothesis. Our finding that estrogen treatment is associated with diminution of brain Aβ levels suggests that modulation of Aβ metabolism is one of the ways by which estrogen prevents and/or delays the onset of AD in postmenopausal women.

It remains possible that the estrogen-associated preservation of cognitive function in post-menopausal women results from multiple activities of estrogen, such as providing trophic support for basal forebrain cholinergic neurons (Luine, V., Exp. Neurol., 1985, 89:484-490), stimulation of neurite outgrowth and synaptogenesis (McEwen and Woolley, Exp. Gerontol., 1994, 29:431-436), stimulation of apolipoprotein E expression (Srivastava et al., J. Biol. Chem., 1997, 272:3360-33366; Stone et al., Exp. Neurol., 1997, 143:313-318) and/or protection of neurons from oxidative stress and Aβ induced toxicity (Gridley et al., Brain Res., 1997, 778:158-165). However, these are the first data showing that estrogen has an effect on Aβ levels in the brain of living animals.

The availability of in vivo systems of the invention enable the investigation of each of these neuroactivities of estrogen under physiological (i.e., guinea pigs) and pathophysiological (i.e., plaque-forming transgenic mice) conditions, and will facilitate the experimental dissection of this problem.

### Example 2

### Ovariectomy and 17β-Estradiol Modulate the Levels of Amyloid β Peptides in APP Transgenic Rodents

This Example shows that estrogen positively impacts Aβ production in rodents made transgenic for human APP, and preferably for presenilin 1 or presenilin 2 as well.

### Materials and Methods

Transgenic APP and APP/PS rodents. Transgenic animals relevant to Alzheimer's Disease have been reviewed (Seabrook and Rosahl, Neuropharmacology, 1999, 38:1-17; see, Detailed Description, supra). Both mice and rats have been made transgenic for APP, for PS1 and for both genes, and with wild-type and FAD mutant forms of the genes, and with wild-type and FAD mutant forms of the genes. One group of these animals is ovariectomized. 17β-Estradiol (E2) is purchased from Sigma (St. Louis, Mo.). Animals are fed ad libitum in a controlled lighting environment, using a casine-based, soy-free diet, as described in Example 1. After 8 weeks on a soy-free diet, animals are divided into 4 groups: i) intact animals; ii) ovx animals; iii) ovx animals that receive a low dose E2 treatment; and iv) ovx animals that receive a high dose E2 treatment. E2 is administered per os by powdering the hormone in the soy-free chow. All animals are weighed at the beginning prior to treatment. Average daily food intake is determined prior to treatment as well. Animals receive soy-free food supplemented with E2 for 10 days; control animals receive the food free of the E2 supplementation.

Tissue collection. After treatment, all animals are sacrificed by decapitation. Trunk blood is collected for determination of E2 levels. Uteri are removed and weighed to establish the presence of atrophy due to estrogen deficiency or E2-induced hypertrophy. Brains are immediately removed and the cerebellum dissected away. The brain is divided into hemispheres which are snap-frozen and stored at −80° C.

Preparation of Brain Extracts. Sample Proteins from Brains are Recovered Using the protocol described in Example 1. Protein concentration are determined using BCA reagent assay kits (Pierce, Rockford, Ill.).

Detection of sAPPα, flAPP, Aβ40 and Aβ42. Because these animals are transgenic for human APP, well characterized Aβ antibodies can be used to study the effects of estrogen on APP metabolism. Soluble APP (sAPPα) is detected by Western blotting of proteins from DEA extracts using monoclonal antibody 6E10, as described in Example 1. Full-length==APP (flAPP) levels are determined by immunoblotting of SDS extracts using antibody 369, as described in Example 1. Levels of Aβ40 and Aβ42 are determined by specific ELISA, as described in Example 1.

In all experiments, animals are coded prior to tissue collection and the treatment status of each animal is unknown to the investigators at the time of assays.

Statistical analysis. For each analyzed perimeter, the values obtained for the intact group are either ovariectomized, E2 treated groups are compared to the values obtained from the ovariectomized, using, for example, a one tailed student's p test. Differences in total Aβ levels are evaluated between the intact group and the ovariectomized group, and between the ovariectomized group and the low and high dose E2 groups. These data can also be assessed using the Mann-Whitney non-parametric test.

### Results and Discussion

Oral administration of E2 leads to uterine hypertrophy and a dramatic increase in serum E2 levels in ovariectomized animals compared to the untreated ovariectomized group.

Ovariectomization results in increased levels of Aβ. E2 treatment correlates with a decrease in the levels of brain Aβ in ovariectomized animals, approaching the levels found in intact animals. These data are obtained in both short term and long term experiments.

These data confirm that levels of Aβ in brain are under the control of gonadal hormones, particularly female gonadal hormones.

### Example 3

### Use of Ovariectomized Animals to Test Aβ Inhibitory Compounds

The ovariectomized guinea pig model described in Example 1 or the overiectomized transgenic rodent model described in Example 2 can be used to screen for compounds or, more optimally, to evaluate candidate compounds obtained from screens for the ability to affect Aβ levels in the brains of these animals. Aβ levels can be evaluated using the methods described in Examples 1 and 2, supra.

Gonadal hormones are one type of compound that can be tested this way. These hormones can be administered per os as well as parentarelly. Other compounds suspected of affecting Aβ levels also can be tested, as the use of ovariectomized animals provides a model with an increased window or signal to noise ratio.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all sizes and all weight or mass values are approximate, and are provided for description.

Patents, patent applications, procedures, and publications cited throughout this application are incorporated herein by reference in their entireties.

1-30. (canceled)

31. A method for reducing a level of amyloid-β (Aβ) peptides in vivo, comprising determining an amount of an estrogen compound effective to reduce the level of amyloid-β (Aβ) peptides in the brain of an animal without affecting soluble APP levels, based upon an effective amount determined in an ovariectomized non-human animal wherein the in vivo level of Aβ peptides in the brain of an animal is reduced without affecting soluble APP levels in the brain.

32. The method of claim 31, wherein the estrogen compound is 17β-estradiol.

33. The method of claim 31, wherein the estrogen compound is a composition of conjugated equine estrogen.

34. The method of claim 31, wherein the Aβ peptides comprise Aβ42 and Aβ40, wherein the effective amount of estrogen compound reduces the ratio of Aβ42 to Aβ40.

35. The method of claim 31, wherein the Aβ peptides are Aβ42 peptides.

36. A method for evaluating the ability of a test compound to reduce a level of amyloid β (Aβ) in vivo in the brain, which method comprises the administration of the test compound to an ovariectomized non-human animal and comparing the level of Aβ with that of an untreated ovariectomized animal.

37. The method of claim 36, wherein the animal is a guinea pig.

38. The method of claim 36, wherein the animal is a transgenic rodent that expresses a human amyloid precursor protein.

39. The method of claim 38, wherein the animal is a double transgenic rodent that also expresses a presenilin protein.

40. The method of claim 36, wherein the test compound is an estrogen compound.

41. A method for evaluating the ability of a test compound to reduce a level of Aβ in vivo in the brain, which method comprises comparing the level of Aβ of an ovariectomized non-human animal selected from the group consisting of a guinea pig and a transgenic rodent that expresses human amyloid precursor protein treated with the test compound to the level of Aβ in an ovx non-human control animal, wherein a reduction of the level of Aβ in brain of the animal treated with the test compound compared to the level of Aβ in the brain of a control animal indicates the ability of the test compound to reduce the level of Aβ in vivo in the brain of an animal.

42. A method for evaluating the ability of a test compound to reduce a ratio of amyloid β (Aβ)42 to Aβ40 in vivo which method comprises the administration of the test compound to an ovariectomized non-human animal and the comparison of the levels of (Aβ)42 and Aβ40 in the brain with those levels of (Aβ)42 and Aβ40 in the brain of an untreated ovariectomized animal.

43. The method of claim 42, wherein the animal is a guinea pig.

44. The method of claim 42, wherein the compound is an estrogen compound.

45. The method of claim 44, wherein the estrogen compound is 17β-estradiol.

46. A method for evaluating the ability of a test compound to reduce a level of amyloid-β (Aβ) peptides in vivo in a subject, comprising administering an amount of the estrogen compound effective to reduce the Aβ level in the brain of an ovariectomized non-human animal to delay or prevent the onset of, or ameliorate, a disease or disorder associated with amyloidosis without affecting soluble APP levels in a subject; wherein the subject has an increased risk for developing or shows a symptom of the disease or disorder associated with amyloidosis.

47. The method of claim 46, wherein the estrogen compound is 17β-estradiol.

48. The method of claim 46, wherein the estrogen compound is administered daily for at least ten days.

49. The method of claim 46, wherein the estrogen compound comprises a controlled release device.

50. The method of claim 46, wherein the disease or disorder associated with amyloidosis is Alzheimer's disease.

51. The method of claim 46, wherein the estrogen compound has the property of reducing a ratio of Aβ42 to Aβ40 in the subject.

52. A method for predicting an increased likelihood of amyloidosis in a subject, which method comprises observing a reduction in a level of an estrogen compound in a biological sample from the subject compared to a normal level or a level in a biological sample from the subject at an earlier time.

53. The method of claim 52, wherein the estrogen compound is estrogen P17.

54. The method of claim 52, wherein the estrogen compound is an aromatizable androgen.

55. The method of claim 52, wherein the amyloidosis comprises deposition of Aβ peptides.

56. The method of claim 55, which comprises predicting an increased likelihood of developing Alzheimer's disease.
